(19) United States
(12) Patent Application Publication
(10) Pub. No.: US 2006/0229524 A1
(43) Pub. Date: Oct. 12, 2006

(54) Method for the accurate placement of EKG electrodes

(76) Inventor: Alden Ozment, Longview, TX (US)

Correspondence Address: STITES & HARBISON, PLLC, 400 W MARKET ST, SUITE 1800, LOUISVILLE, KY, 40202-3352, US

(21) Appl. No.: 11/358,156

(22) Filed: Feb. 21, 2006

Related U.S. Application Data
(60) Provisional application No. 60/669,676, filed on Apr. 8, 2005.

(51) Int. Cl. A61B 5/0402

(52) U.S. Cl. 600/509

(57) ABSTRACT

A method is provided for the accurate placement and replacement of EKG electrodes on a patient. In accordance with the invention, the method comprises marking the position of the EKG electrodes with a removable tattoo so that on a subsequent run the electrodes can be replaced at their original locations as marked. The method also allows an experienced operator to first mark the proper location so less experienced operators can correctly position the electrodes on a patient.

### REFERENCE TO RELATED APPLICATIONS

This application claims the benefit of the filing date of provisional application Ser. No. 60/669,676, filed Apr. 8, 2005, entitled METHOD FOR ACCURATE PLACEMENT OF EKG ELECTRODES that is incorporated by reference herein.

### BACKGROUND OF THE INVENTION

The electrocardiogram (EKG) determines the electrical activity of the heart. The EKG is used to measure the rate and regulation of the heartbeats as well as the size and position of the chambers of the heart. The EKG apparatus comprises an instrument for receiving signals from a plurality of electrodes which detect and transmit to the instrument voltage signals produced by the heart as it functions. A conductive gel is placed at the location of the electrode to aid in conduction of the voltage signal to the electrode and an adhesive strip is used to retain the electrode in its location. The instrument produces a readout signal that can be shown on an oscilloscope or, more preferably, printed as a chart on paper, which is referred to as a strip. For normal heart monitoring such as during a conventional physical, 3 to 4 electrodes are attached at selected locations on the patient's body to measure the voltage signal produced from various parts of the heart. For diagnostic work as many as 12 to 15 electrodes may be applied to the patient's body. The signal is converted by the EKG instrument into a wave form that can be interpreted to determine if the heart is functioning normally or if it exhibits a pattern distinctive of diseases of the heart such as, for example, enlargement of the heart, a myocardial infarction, disrhythmia, left and right hypertrophy and the like.

Location of the electrodes is critical and can determine the results obtained. For example, when monitoring a patient over a period of hours or days it will be common practice to remove the electrodes between runs since the gels and adhesives utilized to improve conductivity and to retain the electrodes in place can irritate the skin of sensitive individuals. Consequently the electrodes are normally removed between runs and then replaced for each subsequent run. If the electrodes are not accurately replaced, the results obtained can vary and the results obtained between runs become meaningless.

Accordingly a simple, efficient and inexpensive method for accurately replacing the electrodes on a patient is highly desirable.

### SUMMARY OF THE INVENTION

A method is provided for the accurate placement of EKG electrodes and the accurate replacement of electrodes during a sequence of EKG procedures on the same patient. In accordance with the invention, a method is provided for marking the position of the EKG electrodes so that on a subsequent run the electrodes can be replaced at their original locations as marked.

### DESCRIPTION OF THE INVENTION

In carrying out an EKG procedure, electrodes of an EKG instrument are placed on various positions on the patient's body, normally on the patient's torso and extremities, to record the electrical activity of the patient's heart. Signals from the electrodes are recorded by the instrument for viewing on an oscilloscope or for printing on a chart. The electrodes are conventionally held in position on the patient by means of adhesive tape or an equivalent adhesive system. In addition there is normally a conductive gel applied to the patient's body at the location of the electrode to improve transmission of the voltage signals from the heart to the electrode.

In carrying out a diagnosis, a sequence of several EKG procedures may be required over a period of hours and in some cases over a period of days. To reduce the discomfort to the patient, the electrodes are normally removed from the body in order to avoid irritation by the adhesive or the conductive gel. For a subsequent EKG run it is essential that the electrodes be replaced in their original locations because variations in the placement of the electrodes can result in variations in the results and make the diagnosis inaccurate. This invention provides a simple but efficient and inexpensive method for accurately replacing and locating the EKG electrodes on the patient.

The method of the invention provides for the placement of a temporary mark at each location of an electrode. The mark can be placed adjacent the location of the electrode or, as described below, can be formed directly at the location from indicia incorporated in the adhesive or the conductive gel that leaves a mark when the electrode is removed. The mark is a temporary tattoo that can remain on the patient's body for sufficient time to complete the sequence, often as long as 2 to 3 days, to indicate the original position of the electrodes. The mark can be produced by a water-actuated decal which may indicate not only the location of the mark but other informational indicia, for example to identify the particular type of electrode that is positioned at the location. Water activated decals are commercially available and can be customized as desired. The decals can be readily removed with soap and water upon completion of the EKG procedures.

In another embodiment of the invention, the marking material is incorporated in the conductive gel or the adhesive holding the electrode on the body. For example, the marking material can be incorporated in the adhesive portion of tape used to secure the electrode to the patient. The marking material is readily incorporated in the conductive gel. Upon removal of the adhesive tape or other adhesive system and the electrode, a sufficient amount of the marking material remains on the patients skin along with adhesive remaining after the tape or adhesive system has been removed or with the remaining conductive gel if contained in the gel to designate the location of the electrode.

Preferably the marking material is a vegetable dye. Vegetable dyes are approved by the FDA and are non-toxic. Longer lasting marks can be produced when using henna as the marking material. The marks will commonly wear off after a few hours and in some cases as long as 2 to 4 days. The marks can be more quickly removed with baby oil or isopropyl alcohol.

As described herein, the marks are applied in the area of already positioned electrodes so that on subsequent EKG runs the electrodes can be accurately relocated at the positions on the patient they originally held in the previous EKG run. However, it will be understood that the marks can be applied on the patient prior to the attachment of the electrodes such as in the case where experienced EKG operators are available to mark the positions for placement of the electrodes by less experienced operators. Marks applied to indicate electrode location prior to attachment of the electrodes is a highly useful tool for training EKG operators.

Having described the invention I claim:

1. In performing an electrocardiogram procedure utilizing electrodes to sense electrical signals: from the heart, a method for the accurate placement of an electrode on a patient comprising the step of producing a temporary mark on a patient's body to indicate the location at which the electrode is to be placed.

2. The method of claim 1 wherein the temporary mark remains at the location of an electrode after the removal of the electrode.

3. The method of claim 1 wherein a temporary mark is placed on the patient prior to the attachment of an electrode.

4. The method of claim 1 wherein the temporary mark is produced by a decal.

5. The method of claim 2 wherein the decal is a water-activated decal

6. The method of claim 1 wherein the temporary mark is produced by a marking material incorporated in an adhesive composition for securing the electrode on the patient.

7. The method of claim 4 wherein the temporary mark is produced by a vegetable dye incorporated in the adhesive composition.

8. The method of claim 1 wherein the temporary mark is produced by a marking material incorporated in the conductive gel composition.

9. The method of claim 6 wherein the temporary mark is produced by a vegetable dye incorporated in the conductive gel.

10. The method of claim 1 wherein the temporary mark includes indicia to identify the electrode that is at that location.

11. In performing a sequence of electrocardiogram procedures the step of placing a temporary mark at the location of an electrode during the first procedure in the sequence and thereafter utilizing the temporary mark to locate that electrode during subsequent electrocardiogram procedures.
